(19) United States
(12) Patent Application Publication
(10) Pub. No.: US 2013/0143881 A1
(43) Pub. Date: Jun. 6, 2013

(54) Hydrated Crystalline Forms of N-[3-fluoro-4-(oxy)phenyl]-N'-(4-fluorophenyl)cyclopropane-1,1-dicarboxamide

(71) Applicants: Hilary Cannon, Hitchin (GB); Feirong Kang, South San Francisco, CA (US); Frederick G. Vogt, South San Francisco, CA (US)

(72) Inventors: Hilary Cannon, Hitchin (GB); Feirong Kang, South San Francisco, CA (US); Frederick G. Vogt, South San Francisco, CA (US)

(73) Assignee: Exelixis, Inc., South San Francisco, CA (US)

(21) Appl. No.: 13/634,275

(22) PCT Filed: Mar. 11, 2011
(86) PCT No.: PCT/US11/28035

§ 371 Date: Jan. 18, 2013

Related U.S. Application Data
(60) Provisional application No. 61/313,192, filed on Mar. 12, 2010.

(51) Int. Cl. C07D 413/12

(52) U.S. Cl. CPC C07D 413/12
USPC 514/235.2; 544/128

(57) ABSTRACT

This invention relates crystalline hydrates of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide, Compound (I). The invention provides methods for treatment of cancer by exploiting the modulation of protein kinase activity. The invention also provides pharmaceutical compositions containing a crystalline hydrate of Compound (I) and a pharmaceutically acceptable excipient.

### CROSS-REFERENCE TO RELATED APPLICATIONS

This application claims priority under 35 U.S.C. §119 to U.S. Application Ser. No. 61/313,192, filed Mar. 12, 2010, the entire contents of each of which are incorporated herein by reference.

### FIELD OF THE INVENTION

This invention relates to hydrated crystalline forms of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide. The invention also relates to pharmaceutical compositions containing crystalline forms of the invention. The invention further relates to methods of treating cancer by inhibiting, regulating and/or modulating kinase signal transduction using crystalline hydrates of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide.

### BACKGROUND OF THE INVENTION

Traditionally, dramatic improvements in the treatment of cancer are associated with identification of therapeutic agents acting through novel mechanisms. One mechanism that can be exploited in cancer treatment is the modulation of protein kinase activity because signal transduction through protein kinase activation is responsible for many of the characteristics of tumor cells. Protein kinase signal transduction is of particular relevance in, for example, renal, gastric, head and neck, lung, breast, prostate, and colorectal cancers; hepatocellular carcinoma; as well as in the growth and proliferation of brain tumor cells.

Protein kinases can be categorized as receptor type or non-receptor type. Receptor-type tyrosine kinases are comprised of a large number of transmembrane receptors with diverse biological activity. For a detailed discussion of the receptor-type tyrosine kinases see Plowman et al., DN&P 7(6): 334-339, 1994. Since protein kinases and their ligands play critical roles in various cellular activities, deregulation of protein kinase enzymatic activity can lead to altered cellular properties, such as uncontrolled cell growth associated with cancer. In addition to oncological indications, altered kinase signaling is implicated in numerous other pathological diseases, including, for example, immunological disorders, cardiovascular diseases, inflammatory diseases, and degenerative diseases. Therefore, protein kinases are attractive targets for small molecule drug discovery. Particularly attractive targets for small-molecule modulation with respect to antiangiogenic and antiproliferative activity include receptor type tyrosine kinases c-Met, KDR, c-Kit, Axl, fit-3, and fit-4.

The kinase c-Met is the prototypic member of a subfamily of heterodimeric receptor tyrosine kinases (RTKs) which include Met, Ron and Sea. The endogenous ligand for c-Met is the hepatocyte growth factor (HGF), a potent inducer of angiogenesis. Binding of HGF to c-Met induces activation of the receptor via autophosphorylation resulting in an increase of receptor dependent signaling, which promotes cell growth and invasion. Anti-HGF antibodies or HGF antagonists have been shown to inhibit tumor metastasis in vivo (See: Maulik et al Cytokine & Growth Factor Reviews 2002 13, 41-59). c-Met overexpression has been demonstrated on a wide variety of tumor types including breast, colon, renal, lung, squamous cell myeloid leukemia, hemangiomas, melanomas, astrocytomas, and glioblastomas. Additionally activating mutations in the kinase domain of c-Met have been identified in hereditary and sporadic renal papilloma and squamous cell carcinoma. (See, e.g., Maulik et al., Cytokine & growth Factor reviews 2002 13, 41-59; Longati et al., Curr Drug Targets 2001, 2, 41-55; Funakoshi et al., Clinica Chimica Acts 2003 1-23).

Inhibition of epidermal growth factor (EGF), vascular endothelial growth factor (VEGF) and ephrin signal transduction will prevent cell proliferation and angiogenesis, two key cellular processes needed for tumor growth and survival. Kinase KDR (refers to kinase insert domain receptor tyrosine kinase) and fit-4 (fms-like tyrosine kinase-4) are both VEGF receptors. Inhibition of EGF, VEGF and ephrin signal transduction will prevent cell proliferation and angiogenesis, two key cellular processes needed for tumor growth and survival. Matter. A., Tumor angiogenesis as a therapeutic target, Drug Discovery Today (2001), 6(19), 1005-1024. EOF and VEGF receptors are desirable targets for small molecule inhibition. All members of the VEGF family stimulate cellular responses by binding to tyrosine kinase receptors (the VEGFRs) on the cell surface, causing them to dimerize and become activated through transphosphorylation. The VEGF receptors have an extracellular portion having immunoglobulin-like domains, a single transmembrane spanning region and an intracellular portion containing a split tyrosine-kinase domain. VEGF binds to VEGFR-1 and VEGFR-2. VEGFR-2 is known to mediate almost all of the known cellular responses to VEGF.

Kinase c-Kit (also called stem cell factor receptor or steel factor receptor) is a type 3 receptor tyrosine kinase (RTK) belonging to the platelet-derived growth factor receptor subfamily. Overexpression of c-Kit and c-Kit ligand has been described in variety of human diseases including human gastrointestinal stromal tumors, mastocytosis, germ cell tumors, acute myeloid leukemia (AML). NK lymphoma, small-cell lung cancer, neuroblastomas, gynecological tumors and colon carcinoma. Moreover, elevated expression of c-Kit may also relate to the development of neoplasia associated with neurofibromatosis type 1 (NF-1), mesenchymal tumors GISTs and mast cell disease, as well as other disorders associated with activated c-Kit.

Kinase Flt-3 (fms-like tyrosine kinase-3) is constitutively activated via mutation, either in the juxtamembrane region or in the activation loop of the kinase domain, in a large proportion of patients with AML (Reilly, Leuk. Lymphoma 2003 44: 1-7).

Accordingly, small-molecule compounds that specifically inhibit, regulate, and/or modulate the signal transduction of kinases, particularly including c-Met, VEGFR2. KDR, c-Kit, Axl, fit-3, and flt-4 described above, are particularly desirable as a means to treat or prevent disease states associated with abnormal cell proliferation and angiogenesis. One such small-molecule is N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide, Compound (I), which has the chemical structure:

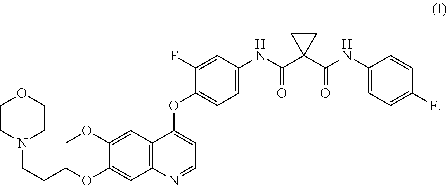 [SMILES: COc1cc2c(Oc3ccc(NC(=O)C4(C(=O)Nc5ccc(F)cc5)CC4)cc3F)ccnc2cc1OCCCN1CCOCC1]

WO 2005-030140 describes the synthesis of Compound (I) (Examples 25, 30, 36, 42, 43 and 44) and also discloses the therapeutic activity of this molecule to inhibit, regulate and/or modulate the signal transduction of kinases, (Assays, Table 4, entry 312). Compound (I) has been measured to have a c-Met IC₅₀ value of about 0.6 nanomolar (nM). PCT/US09/064,341, which claims priority to U.S. provisional application 61/199,088, filed Nov. 13, 2008, describes a scaled-up synthesis of Compound (I).

Although therapeutic efficacy is the primary concern for a therapeutic agent, the solid-state form can be equally important to its development. Generally, the drug developer endeavors to discover a crystalline form that possesses desirable properties such as satisfactory water-solubility (including rate of dissolution), storage stability, hygroscopicity, formulatability, and reproducibility, all of which can impact the processability, manufacture, and/or bioavailability of the drug. Accordingly, discovery of one or more crystalline forms that possess some or all of these desired properties is vital to drug development.

### SUMMARY OF THE INVENTION

This invention relates to a crystalline hydrate of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide, Compound (I) that exists as a variable hydrate with several states of hydration. The invention provides methods for treatment of cancer by exploiting the modulation of protein kinase activity. As discussed above, signal transduction through protein kinase activation is responsible for many of the characteristics of tumor cells. Protein kinase signal transduction is of particular relevance in, for example, renal (e.g. papillary renal cell carcinoma), gastric (e.g. metastatic gastric carcinoma), head and neck (e.g. squamous cell carcinoma), lung, breast, prostate, and colorectal cancers, quamous cell myeloid leukemia, hemangiomas, melanomas, astrocytomas, glioblastomas, hepatocellular carcinoma, hereditary and sporadic renal papilloma, as well as in the growth and proliferation of brain tumor cells.

Accordingly, the invention also relates to methods of treating cancer. These methods comprise administering to a subject in need thereof therapeutically effective amounts of at least one crystalline hydrate of Compound (I).

In another embodiment, the invention provides methods of treating diseases or disorders associated with uncontrolled, abnormal, and/or unwanted cellular activities. These methods comprise administering to a subject, in need thereof, therapeutically effective amounts of at least one crystalline hydrate of Compound (I).

The invention further provides pharmaceutical compositions containing therapeutically effective amounts of at least one crystalline hydrate of Compound (I) and a pharmaceutically acceptable excipient.

### BRIEF DESCRIPTION OF THE FIGURES

FIG. 1-A shows the sorption and desorption curves of the Gravimetric Vapor Sorption Study (GVS) of Compound (I) crystalline hydrate from Example 1.1.1.

FIG. 1-B shows the XRPD pattern for Compound (I) crystalline hydrate from Example 1.1.1.

FIG. 1-C shows the DSC thermogram of Compound (I) crystalline hydrate from Example 1.1.1.

FIG. 1-D shows the TGA thermogram of Compound (I) crystalline hydrate from Example 1.1.1.

FIG. 1-E shows the solid state ¹³C NMR spectra of Compound (I) crystalline hydrates from Example 1.1.2.

FIG. 1-F shows the solid state ¹⁹F NMR spectra of Compound (I) crystalline hydrates from Example 1.1.2.

FIG. 1-G shows the Raman spectra of Compound (I) crystalline hydrates from Example 1.1.2. Arrows denote subtle changes in the spectra related to hydration state. FIG. 1-G(a) shows the spectrum from 3700 cm⁻¹ to 200 cm⁻¹. FIG. 1-G(b) shows a blow up of the spectrum from 1700 cm⁻¹ to 1100 cm⁻¹.

FIG. 1-H shows the XRPD patterns of Compound (I) crystalline hydrates under relative humidity conditions from Example 1.1.3. In (a), the full diffraction patterns are shown. In (b), expanded regions highlighting peak shifts are shown. In both (a) and (b), diffraction patterns are shown for the following conditions (from top to bottom): the initial condition of 40 percent RH, immediately after vacuum is applied (to reach nearly 0 percent RH), 125 minutes after vacuum is applied, and after the material is returned to 40 percent RH.

### DETAILED DESCRIPTION OF THE INVENTION

### Crystalline Hydrates of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide, Compound (I)

The invention relates to a crystalline hydrate of Compound (I) that exists as a variable hydrate with several hydration states, varying from about 0.1 molar equivalent of water to about 1 molar equivalent of water relative to Compound (I). The Examples below describe these crystalline hydrates of Compound (I) according to the invention including their preparation and characterization. These crystalline forms are variable hydrates, also known as isomorphic desolvates and channel hydrates, where the degree of hydration ranges from nearly anhydrous to an upper stoichiometric limit approximately equal to a monohydrate. (Stephenson, G. A.; Groleau, E. G.: Kleeman, R. L; Xu, W.; Rigsbee, D. R. J. Pharm. Sci. 1998, 87, 536-42). The solid state of a compound can be characterized by various physical properties such as solubility, melting point, x-ray powder diffraction, solid state NMR spectroscopy, and Raman spectroscopy. Generally, different crystalline forms of a compound can be identified, or characterized, one from the other by comparing their respective analytical data, such as their XRPD patterns or solid state NMR peaks. In the present instance, the hydrate states of the present invention result in solid state characterization data that contain similarities consistent with a single form. These similarities ae evidenced by the peaks in Table 1, which shows characteristic peaks from the XRPD patterns, the solid state NMR spectra, and the Raman spectra, that are common to the range of hydration states.

TABLE 1

| | | Raman | XRPD |
|---|---|---|---|
| ¹³C solid-state NMR | ¹⁹F solid-state NMR | spectroscopy | (degrees 2 |
| (ppm, ±0.2 ppm, | (ppm, ±0.4 ppm, | (Raman shift, | theta ± |
| from TMS) | from CFCl₃) | cm⁻¹ ± 2 cm⁻¹) | 0.2 °2 theta) |
| 173.3 | −116.3 | 1623 | 9.0 |
| 160.9 | −125.1 | 1503 | 10.2 |
| 158.6 | | 1436 | 12.0 |
| 155.3 | | 1337 | 15.6 |
| 152.7 | | 901 | 16.2 |
| 149.8 | | 853 | 19.9 |
| 135.4 | | 779 | 20.3 |
| 125.4 | | 744 | 22.1 |
| 100.3 | | 708 | 24.4 |
| 67.1 | | 634 | |
| 54.6 | | | |
| 26.1 | | | |
| 22.6 | | | |

The crystalline hydrates of the present invention may be characterized by these sets of characteristic peaks separately or combinations thereof or subsets thereof. For example, combinations and subsets of peaks that are not subject to interference by common pharmaceutical exicipents may be used to characterize the crystalline hydrates.

Crystalline hydrates of Compound (I) disclosed here may possess advantages vis-á-vis each other and other forms. Such advantages may suggest the use of one form for a particular formulation or processing, or as an intermediate. For example, a crystalline anhydrate of Compound (I) has the propensity to convert to the hydrate of the present invention in water-based formulations.

As shown in the Examples below, a crystalline hydrate of Compound (I) may be prepared by dissolving Compound (I) in an aqueous solvent, and then crystallizing the crystalline hydrate of Compound (I) from the aqueous solution. The aqueous solvent may be water or a combination of water and an organic solvent, for example, a combination of water and acetone. Alternatively, a crystalline hydrate of Compound (I) may be prepared by placing a crystalline hydrate of Compound (I) in a humidity chamber under conditions and for a time sufficient to increase or decrease its degree of hydration. The humidity chamber may be a closed environment with controlled humidity or an open environment where its humidity level is sufficient to cause a change in hydration when a crystalline Compound (I) hydrate is exposed to that open environment.

### Methods of Treatment

As discussed above, Compound (I) possesses beneficial therapeutic properties in its ability to specifically inhibit, regulate and/or modulate the signal transduction of kinases, particularly including c-Met. KDR, c-Kit, Axl, fit-3, and fit-4. This makes Compound (I) particularly desirable as a therapeutic to treat and/or prevent disease states associated with abnormal cell proliferation and angiogenesis.

The invention therefore provides methods for treatment and/or prevention of cancer by exploiting the modulation of protein kinase activity. As discussed above, signal transduction through protein kinase activation is responsible for many of the characteristics of tumor cells. Protein kinase signal transduction is of particular relevance in, for example, renal cancer (e.g. papillary renal cell carcinoma, sporadic renal papilloma), gastric cancer (e.g. metastatic gastric carcinoma), head and neck cancer (e.g. squamous cell carcinoma), lung cancer (e.g. non-small cell lung cancer), breast cancer, prostate cancer, and colorectal cancers, quamous cell myeloid leukemia, hemangiomas, melanomas, brain cancers (e.g. astrocytomas, glioblastomas), and hepatocellular carcinoma.

Accordingly, the invention relates to a method of treating and/or preventing cancer. The method comprises administering to a subject, in need thereof, a therapeutically effective amount of a crystalline hydrate of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide. Compound (I), according to the invention. The crystalline hydrate of Compound (I) administered may be in any of the crystalline hydrates of the invention and mixtures of crystalline hydrates. The subject to be treated is generally a mammal and most often a human. The cancer being treated is preferably one discussed above, such as renal cancer, gastric cancer, head and neck cancer, lung cancer, breast cancer, prostate cancer, colorectal cancer, squamous cell myeloid leukemia, hemangiomas, melanomas, astrocytomas, glioblastomas, hereditary and sporadic renal papilloma, squamous cell carcinoma, and brain tumors but may be any form of cancer for which crystalline hydrates of Compound (I) according to the invention have efficacy.

### Pharmaceutical Compositions of the Invention

The invention relates to pharmaceutical compositions comprising a therapeutically effective amount of at least one crystalline hydrate of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide, Compound (I), according to the invention and at least one pharmaceutically acceptable carrier, (also known as a pharmaceutically acceptable excipient). As discussed above, the crystalline hydrates of Compound (I) are therapeutically useful for the treatment and/or prevention of disease states associated with abnormal cell proliferation and angiogenesis. The crystalline hydrates of Compound (I) possess therapeutic activity to inhibit, regulate and/or modulate the signal transduction of kinases such as described in WO2005-030140. Pharmaceutical compositions for the treatment of those disease states contain a therapeutically effective amount of at least one crystalline hydrate of Compound (I) according to the invention to inhibit, regulate and/or modulate the signal transduction of kinases as appropriate for treatment of a patient with the particular disease. A pharmaceutical composition of the invention may be in any pharmaceutical form which contains a crystalline hydrate of Compound (I) according to the invention. The pharmaceutical composition may be, for example, a tablet, capsule, liquid suspension, injectable, topical, or transdermal. The pharmaceutical compositions generally contain about 1 percent to about 99 percent by weight of at least one crystalline hydrate of Compound (I) of the invention and 99 percent to 1 percent by weight of a suitable pharmaceutical excipient. In one example, the composition will be between about 5 percent and about 75 percent by weight of a crystalline hydrate of Compound (I) of the invention, with the remainder of the composition being suitable pharmaceutical excipients or other adjuvants, as discussed below.

A “therapeutically effective amount of a crystalline hydrate of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide” according to the invention sufficient to inhibit, regulate and/or modulate the signal transduction of kinases” (discussed here concerning the pharmaceutical compositions) refers to any amount sufficient to treat a patient suffering from any of a variety of cancers associated with abnormal cell proliferation and angiogenesis. The actual amount required for treatment of any particular patient will depend upon a variety of factors including the disease state being treated and its severity; the specific pharmaceutical composition employed; the age, body weight, general health, sex and diet of the patient; the mode of administration; the time of administration; the route of administration; and the rate of excretion of the crystalline hydrate of Compound (I) according to the invention; the duration of the treatment; any drugs used in combination or coincidental with the specific compound employed; and other such factors well known in the medical arts. These factors are discussed in Goodman and Gilman's “The Pharmacological Basis of Therapeutics”. Tenth Edition, A. Gilman, J. Hardman and L. Limbird, eds., McGraw-Hill Press, 155-173, 2001. The crystalline hydrates of Compound (I) according to the invention, and pharmaceutical compositions containing them, may be used in combination with anticancer or other agents that are generally administered to a patient being treated for cancer. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is, one containing a crystalline hydrate of Compound (I) of the invention, a carrier should be chosen so as to substantially maintain the particular crystalline hydrate of Compound (I) of the invention. In other words, the carrier should not substantially alter the crystalline hydrate of the compound (I) of the invention. Nor should the carrier be otherwise incompatible with the crystalline hydrate of Compound (I) according to the invention, such as by producing any undesirable biological effect or otherwise interacting in a deleterious manner with any other component(s) of the pharmaceutical composition.

The pharmaceutical compositions of the invention may be prepared by methods known in the pharmaceutical formulation art, for example, see Remington's Pharmaceutical Sciences, 18th Ed., (Mack Publishing Company. Easton, Pa., 1990). In a solid dosage forms, at least one crystalline hydrate of Compound (I) may be admixed with at least one pharmaceutically acceptable excipient such as sodium citrate or dicalcium phosphate or any other excipients known to those of skill in the art, such as: (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. If desired, a pharmaceutical composition of the invention may also contain minor amounts of auxiliary substances including, but not limited to, wetting or emulsifying agents, pH buffering agents, antioxidants, and the like, such as, for example, citric acid, sorbitan monolaurate, triethanolamine oleate, butylalted hydroxytoluene, among others.

Solid dosage forms as described above can be prepared with coatings and shells, such as enteric coatings and others well known in the art. They may contain opacifying agents, and can also be of such composition that they release the active compound or compounds in a certain part of the intestinal tract in a delayed manner. Examples of embedded compositions that can be used are polymeric substances and waxes. The active compounds, at least one crystalline hydrate of Compound (I), can also be in microencapsulated form, if appropriate, with one or more of the above-mentioned excipients.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the invention with, for example, suitable non-irritating excipients or carriers such as cocoa butter, polyethyleneglycol or a suppository wax, which are solid at ordinary temperatures but liquid at body temperature and therefore, melt while in a suitable body cavity and release the active compound therein.

Because the crystalline hydrates of Compound (I) of the invention are maintained during their preparation, solid dosage forms are preferred for the pharmaceutical composition of the invention. Solid dosage forms for oral administration, which includes capsules, tablets, pills, powders, and granules, are particularly preferred. In such solid dosage forms, the active compound is mixed with at least one inert, pharmaceutically acceptable excipient (also known as a pharmaceutically acceptable carrier). Administration of a crystalline hydrate of Compound (I) in pure form, or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. One preferable route of administration is oral administration, using a convenient dosage regimen that can be adjusted according to the degree of severity of the disease-state to be treated.

### EXAMPLES

### Example 1

### Preparation and Physical Characterization of N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide Crystalline Hydrate, Compound (I)

### 1.1.1. Preparation of Compound (I) Crystalline Hydrate.

The hydrate was prepared by adding 4.9614 g of Compound (I) and 50 mL of n-propanol to a 250 mL beaker. The suspension was heated to 90° C. with stirring via a magnetic stir bar at 200 rpm. After 2 hours, the solids were fully dissolved in an amber solution. At the 1 hour and 2 hour timepoints, 10 mL of n-propanol was added to account for evaporative effects and return the volume of the solution to 50 mL. The solution was then hot-filtered through a 1.6 micrometer glass fiber filter. The solution was then allowed to dry overnight in the beaker to a powder, which was then redissolved in 150 mL of a 1:1 mixture of acetone and water, and slurried overnight (16 hours) with a foil lid to prevent evaporation. The slurried solids were then collected by vacuum filtration. The final weight recovered was 3.7324 g (75 percent yield). This batch was stored at ambient conditions for several days prior to analysis.

Karl Fisher water content determinations were performed using a standard procedure. Water content was measured with a Brinkmann KF1V4 Metrohm 756 Coulometer equipped with a 703 Ti stirrer and using Hydranal Coulomat AG reagent. Samples were introduced into the vessel as solids. Approximately 30-35 mg of sample was used per titration. A sample of crystalline Compound (I) prepared in Example 1.1.2 was measured in duplicate and was found to have an average water content be 2.5 percent weight/weight, with each replicate agreeing to within 0.1 percent.

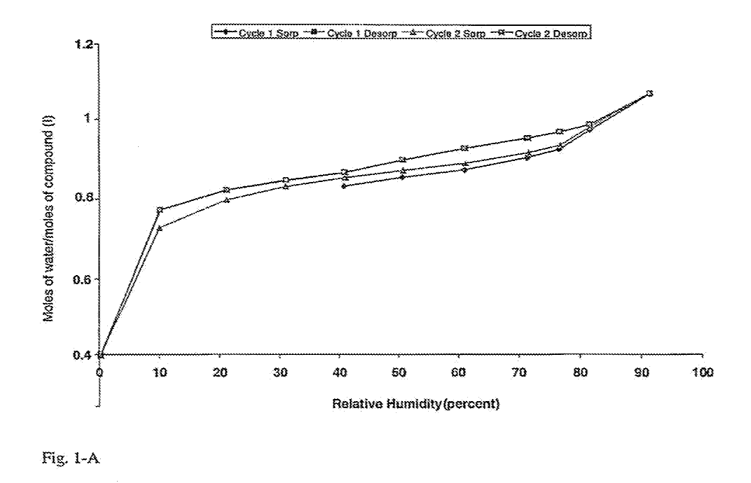

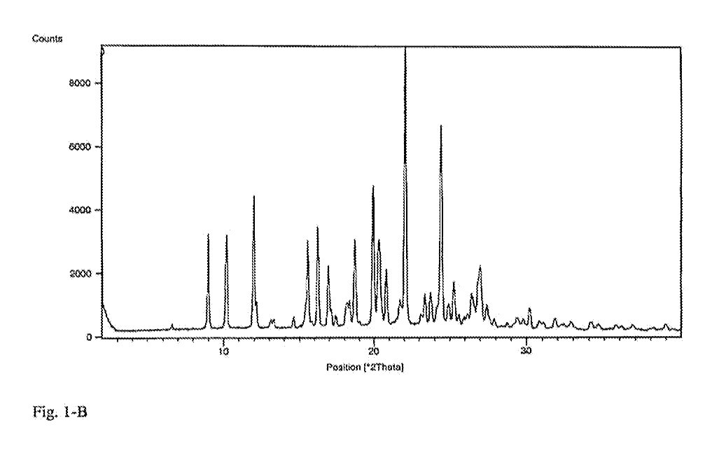

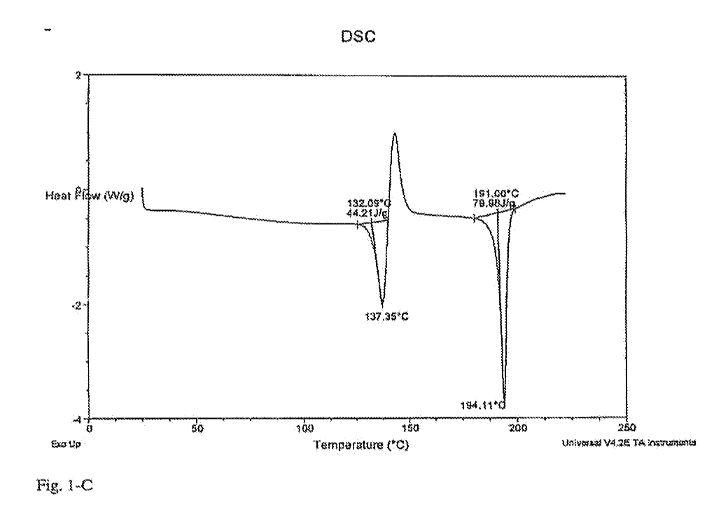

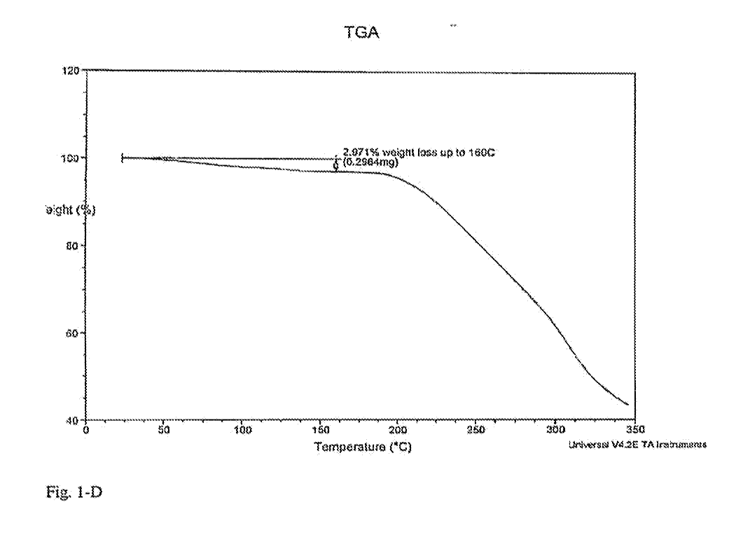

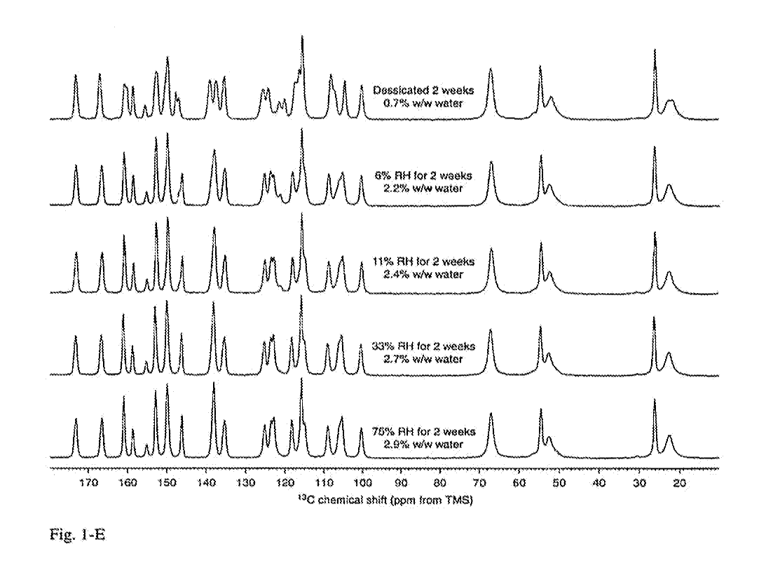

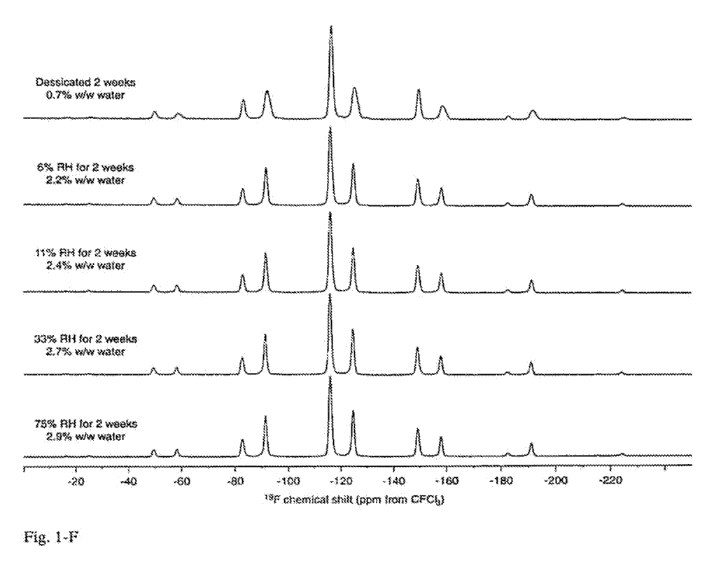

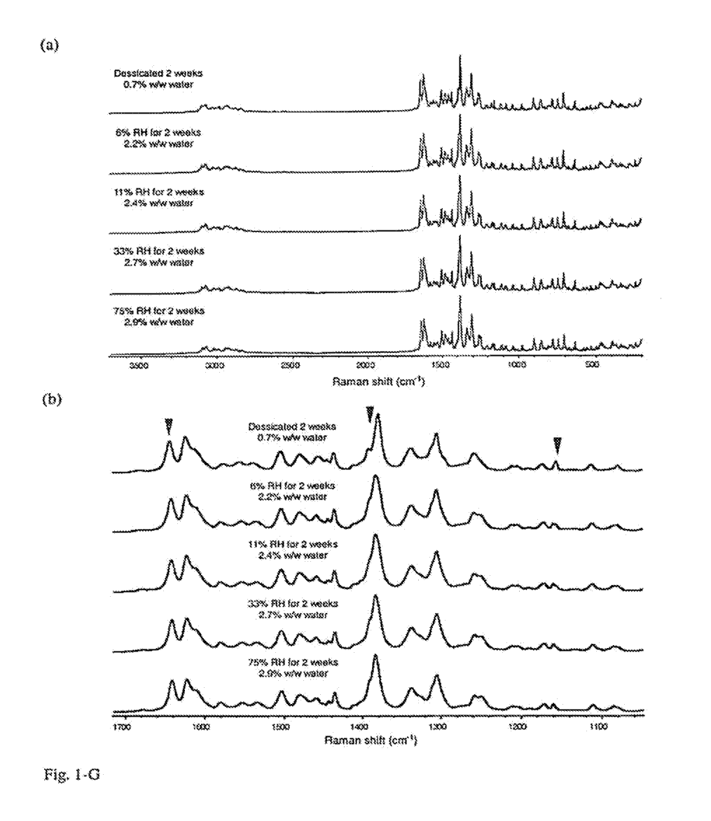

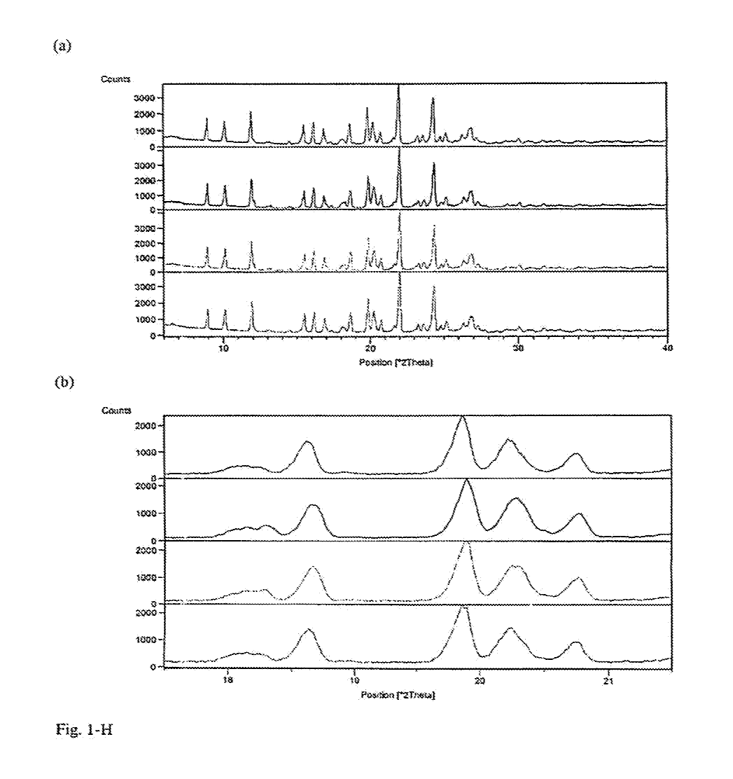

A gravimetric vapor sorption (GVS) study was run using a standard procedure. Samples were run on a dynamic vapor sorption analyzer (Surface Measurement Systems) running DVSCFR software. Sample sizes were typically 10 mg. A moisture adsorption desorption isotherm was performed as outlined below. The standard isotherm experiment, performed at 25° C., is a two-cycle run, starting at 40 percent RH, increasing humidity to 90 percent RH, decreasing humidity to 0 percent RH, increasing humidity again to 90 percent RH, and finally decreasing humidity to 0 percent RH in 10 percent RH intervals. The crystalline Compound (I) prepared in Example 1.1.1 showed a 2.5 percent weight gain at 25° C. and 90 percent humidity. The GVS sorption and desorption curves are shown in FIG. 1-A. The GVS sorption and desorption curves are shown in FIG. 1-A. The GVS results show evidence that the hydrate behaves as an isomorphic desolvate (Stephenson, G. A.; Groleau, E. G.; Kleeman, R. L.: Xu, W.; Rigsbee, D. R. J. Pharm. Sci. 1998, 87, 536-42).

The X-ray powder diffraction pattern of Compound (I) crystalline hydrate prepared in Example 1.1.1 was acquired using a PANalytical X'Pert Pro diffractometer. The sample was gently flattened onto a zero-background silicon insert sample holder. A continuous 2 theta scan range of 2 to 50° was used with a Cu K-alpha radiation source and a generator power of 40 kV and 45 mA. A 2 theta step size of 0.017 degrees/step with a step time of 40.7 seconds was used. Samples were rotated at 30 rpm. Experiments were performed at room temperature and at ambient humidity. FIG. 1-B shows the XRPD pattern for N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide crystalline hydrate from Example 1.1.1. The following peaks at an experimental °2 theta+0.1 °2 theta were identified in the XRPD pattern: 6.6, 9.0, 10.2, 12.0, 12.2, 13.1, 13.3, 14.6, 15.6, 16.2, 17.0, 17.1, 17.4, 18.2, 18.4, 18.7, 20.0, 20.3, 20.8, 21.7, 22.1, 23.1, 23.4, 23.8, 24.2, 24.5, 25.0. Only peaks below 25 °2 theta are given as these are generally preferred for the identification of crystalline pharmaceutical forms. The entire list of peaks, or a subset thereof, may be sufficient to characterize the hydrate of Compound (I).

DSC thermograms were acquired using a TA Instruments Q2000 differential scanning calorimeter. A sample mass of 2.1500 mg of Compound (I) crystalline hydrate prepared in Example 1.1.1 was weighed out directly into an aluminum DSC pan. The pan was sealed by applying pressure by hand and pushing each part the pan together (also known as a loose lid configuration). The temperature was ramped from 25° C. to 225° C. at 10° C./minute. A peak melting temperature of 137.4° C. and a heat flow of 44.2 J/g was measured for the melting endotherm. After the melting event, recrystallization occurs to an anhydrous form, which then melts at 194.1° C. The DSC thermogram is shown in FIG. 1-C. Exothermic events are plotted in the upward direction.

TOA thermograms were acquired using a TA Instruments Q500 Thermogravimetric Analyzer. The sample pan was tared, and 9.9760 milligrams of Compound (I) crystalline hydrate prepared in Example 1.1.1 was placed in the pan. The temperature was ramped from 25° C. to 300° C. at 10° C./minute. A weight loss of 2.97 percent was observed up to 160° C. with an additional weight loss beyond 200° C. from decomposition. The TGA thermogram is shown in FIG. 1-D.

1.1.2. Preparation of Compound (I) Crystalline Hydrate with Different Hydration States.

Five 150 mg aliquots were taken from the crystalline hydrate batch prepared in Example 1.1.1 and were placed in 10 mL screw-top vials. With the vial tops removed, these aliquots were each stored in chambers with desiccant (Dri-Rite®, tricalcium silicate, RH 2-3 percent), saturated lithium bromide (6 percent RH), saturated lithium chloride (11 percent RH), saturated magnesium chloride (33 percent RH), and saturated sodium chloride (75 percent RH). The samples were removed after 2 weeks and immediately sealed with a cap for analysis.

Solid-state NMR spectra of the Compound (I) crystalline hydrates prepared in Example 1.1.2 were acquired using a Bruker Avance 400 triple-resonance spectrometer operating at a ¹H frequency of 399.87 MHz. ¹³C NMR spectra were obtained using a cross-polarization pulse sequence with a Broker 4-mm triple resonance magic-angle spinning probe at a rotor frequency of 8 kHz. A linear power ramp from 75 to 90 kHz was used on the ¹H channel to enhance cross-polarization efficiency. Spinning sidebands were eliminated by a five-pulse total sideband suppression pulse sequence. ¹⁹F spectra were obtained using the same spectrometer and probe, using a cross-polarization pulse sequence and spinning at a rotor frequency of 12.5 kHz. FIG. 1-E shows the solid state ¹³C NMR spectra of the five hydration states of Compound (I) crystalline hydrate prepared in Example 1.1.3. The ¹³C NMR peak positions are reported relative to tetramethylsilnane at 0 ppm (parts per million) and are quoted to a precision of +/−0.2 ppm, because of instrumental variability and calibration. Characteristic peaks for the hydrate from the solid state ¹³C NMR spectra that are common to all of the hydration states include peaks at 173.3, 160.9, 158.6, 155.3, 152.7, 149.8, 135.4, 125.4, 100.3, 67.1, 54.6, 26.1, and 22.6 ppm±0.2 ppm or a subset thereof. In addition to this list, other peaks are observed in FIG. 1-E to shift as the hydration state changes. FIG. 1-F shows the solid state ¹⁹F NMR spectra of the five hydration states of Compound (I) crystalline hydrate prepared in Example 1.1.2. The solid state ¹⁹F NMR spectrum showed peaks −116.3 and −125.1 ppm relative to CFCl₃ and with a precision of ±0.4 ppm, due to instrumental variability and calibration. Both solid state ¹⁹F NMR peaks are considered to be characteristic of hydrate. It is believed that the subtle but readily detectable changes observed in ¹³C and ¹⁹F solid state NMR spectra results suggest that the material desolvates in an isomorphic manner.

The Fourier-transform (FT) Raman spectrum of Compound (I) crystalline hydrate prepared in Example 1.1.2 was acquired using a Thermo Nicolet 960 spectrometer equipped with a liquid nitrogen-cooled germanium detector and a motorized stage accessory with video control. A 1.064 micrometer laser was used with a power setting of 0.55 W. The powdered sample was placed onto a glass microscope slide and placed directly into the beam using the stage. A 1-mm laser spot size was used, and 512 scans were collected at 2 cm⁻¹ resolution. The FT-Raman spectra of the crystalline hydrates of Compound (I) in various hydration states are shown in FIG. 1-G. The following peaks (Raman shift, cm⁻¹+/−2 cm⁻¹) were observed in the FT Raman spectrum to not change over the humidity range explored: 1623, 1503, 1436, 1337, 901, 853, 779, 744, 708, 634.

1.1.3. Characterization of Compound (I) Crystalline Hydrate with Different Hydration States by Variable-Humidity XRPD.

A sample of the Compound (I) crystalline hydrate was prepared using a procedure similar to that in Example 1.1.1. A series of batches of Compound (I) hydrate prepared by slurrying in acetone/water mixtures with activities of 0.3 to 0.9 were combined to create a single batch used for this study. Variable-humidity XRPD was performed on a Bruker D8 Advance X-ray powder diffractometer equipped with an Anton-Par TTK450 temperature stage and SYCOS-H Gas Humidifier. Approximately 30 mg of material was packed into a stainless steel sample holder and gently flattened. The following acquisition parameters were used: Cu K-alpha radiation, 40 mA, 40 kV, continuous scan mode using a step size of 0.017°2 theta over the scan range 2° to 40°2 theta, 0.1 s step time. FIG. 1-H shows the XRPD patterns of crystalline hydrate obtained at the initial condition of 40 percent RH, immediately after vacuum is applied (to reach nearly 0 percent RH), 125 minutes after vacuum is applied, and after the material is returned to 40 percent RH. The XRPD pattern at the initial and final time points matches the pattern collected at ambient conditions and shown in FIG. 1-B. The following peaks at an experimental °2 theta±0.2°2 theta were identified in the XRPD pattern as peaks that did not change as the sample was dried: 9.0, 10.2, 12.0, 15.6, 16.2, 19.9, 20.3, 22.1, and 24.4. The entire list of peaks, or a subset thereof, may be sufficient to characterize the crystalline hydrate of Compound (I).

The claimed invention is:

1. Crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate.

2. The crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1, wherein the degree of hydration ranges from about 0.1 molar equivalent of water to about 1 molar equivalent of water relative to N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide.

3. The crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1 characterized by at least one of:
a solid state ¹³C NMR spectrum with peaks at 173.3, 160.9, 158.6, 155.3, 152.7, 149.8, 135.4, 125.4, 100.3, 67.1, 54.6, 26.1, and 22.6 ppm±0.2 ppm;
a solid state ¹⁹F NMR spectrum with peaks at −116.8 and −128.6 ppm±0.4 ppm relative to CFCl₃;
an X-ray powder diffraction pattern with peaks at 9.0, 10.2, 12.0, 15.6, 16.2, 19.9, 20.3, 22.1, and 24.4 °2 theta±0.22 °2 theta; and
a Ramon spectrum with peaks at 1623, 1503, 1436, 1337, 901, 853, 779, 744, 708, and 634±2 cm⁻¹.

4. The crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 3 characterized by at least two of:
a solid state ¹³C NMR spectrum with peaks at 173.3, 160.9, 158.6, 155.3, 152.7, 149.8, 135.4, 125.4, 100.3, 67.1, 54.6, 26.1, and 22.6 ppm±0.2 ppm;
a solid state ¹⁹F NMR spectrum with peaks at −116.8 and −128.6 ppm±0.4 ppm relative to CFCl₃;
an X-ray powder diffraction pattern with peaks at 9.0, 10.2, 12.0, 15.6, 16.2, 19.9, 20.3, 22.1, and 24.4 °2 theta±0.2 °2 theta; and
a Raman spectrum with peaks at 1623, 1503, 1436, 1337, 901, 853, 779, 744, 708, and 634±2 cm⁻¹.

5. The crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 4, wherein the degree of hydration ranges from about 0.1 molar equivalent of water to about 1 molar equivalent of water relative to N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide.

6. A pharmaceutical composition comprising a therapeutically effective amount of the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1 and a pharmaceutically acceptable excipient.

7. A method of treating cancer, comprising the step of administering to a subject in need thereof a therapeutically effective amount of the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1.

8. The method of claim 7 wherein the subject is a human.

9. The method of claim 7, wherein the cancer being treated is selected from the group consisting of renal cancer, gastric cancer, head and neck cancer, lung cancer, breast cancer, prostate cancer, colorectal cancer, squamous cell myeloid leukemia, hemangiomas, melanomas, squamous cell carcinoma, hepatocellular carcinoma and brain cancer.

10. The method of claim 9, wherein the cancer being treated is selected from the group consisting of papillary renal cell carcinoma, squamous cell carcinoma and metastatic gastric carcinoma.

11. The method of claim 9, wherein the cancer is hepatocellular carcinoma.

12. A method of treating cancer, comprising the step of administering to a subject in need thereof a therapeutically effective amount of the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 3, wherein the cancer being treated is selected from the group consisting of renal cancer, gastric cancer, head and neck cancer, lung cancer, breast cancer, prostate cancer, colorectal cancer, squamous cell myeloid leukemia, hemangiomas, melanomas, squamous cell carcinoma, hepatocellular carcinoma and brain cancer.

13. The method of claim 12 wherein the subject is human.

14. The method of claim 12, wherein the cancer being treated is selected from the group consisting of cell carcinoma, squamous cell carcinoma and metastatic gastric carcinoma.

15. The method of claim 12, wherein the cancer is hepatocellular carcinoma.

16. A method of preparing the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1 comprising the steps of:
dissolving N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide in an aqueous solvent, and
crystallizing the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate from the aqueous solution.

17. A method of preparing the crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate of claim 1 comprising the step of:
placing a crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate in a humidity chamber under conditions and for a time sufficient to increase or decrease the degree of hydration of crystalline N-[3-fluoro-4-({6-(methyloxy)-7-[(3-morpholin-4-ylpropyl)oxy]quinolin-4-yl}oxy)phenyl]-N′-(4-fluorophenyl)cyclopropane-1,1-dicarboxamide hydrate.
